(19) United States
(12) Patent Application Publication
(10) Pub. No.: US 2022/0108371 A1
(43) Pub. Date: Apr. 7, 2022

(54) INFORMATION PROCESSING DEVICE, INFORMATION PROCESSING METHOD, AND PROGRAM

(71) Applicant: TOYOTA JIDOSHA KABUSHIKI KAISHA, Toyota-shi (JP)

(72) Inventors: Toyokazu NAKASHIMA, Nagoya-shi (JP); Takayuki HATORI, Nagakute-shi (JP); Shuichi SAWADA, Nagoya-shi (JP); Daiki KUBO, Toyota-shi (JP); Tomoki ISHIKAWA, Nagoya-shi (JP); Naoya OKA, Nagakute-shi (JP)

(73) Assignee: TOYOTA JIDOSHA KABUSHIKI KAISHA, Toyota-shi (JP)

(21) Appl. No.: 17/474,553

(22) Filed: Sep. 14, 2021

(30) Foreign Application Priority Data
Oct. 1, 2020 (JP) 2020-166991

(51) Int. Cl. G06Q 30/06; G01N 33/497; G05B 13/02

(52) U.S. Cl. CPC G06Q 30/0617; G01N 33/4972; G06Q 10/087; G05B 13/0265; G06Q 30/0633

(57) ABSTRACT

An information processing device including a control unit configured to acquire the output of a smell sensor that senses a user and to estimate the amount of intake of a predetermined article of taste by the user based on the output of the smell sensor.

### CROSS-REFERENCE TO RELATED APPLICATION

This application claims priority to Japanese Patent Application No. 2020-166991 filed on Oct. 1, 2020, incorporated herein by reference in its entirety.

### BACKGROUND

### 1. Technical Field

The present disclosure relates to a technique that assists the user.

### 2. Description of Related Art

A technique for supporting a regular purchase of goods, such as daily goods, is known. For example, Japanese Unexamined Patent Application Publication No. 2016-224650 (JP 2016-224650 A) discloses a system that, upon detecting that the order button on a beacon device is triggered, places an order for goods related to the beacon device. Installing the beacon device, described in JP 2016-224650 A, in a place where goods are stored, for example, near a refrigerator or in a washroom, allows the user to place an order for goods through a simple operation.

### SUMMARY

However, there is room for improvement in the conventional technique in that the user has to manage the stock of, and place an order for, goods by himself or herself. To automate stock management, it is necessary to detect how much predetermined goods have been consumed by the user.

The present disclosure provides a technique for estimating the amount of intake of an article of taste by the user.

A first aspect of the present disclosure relates to an information processing device including a control unit. The control unit is configured to acquire the output of a smell sensor that senses a user and to estimate the amount of intake of a predetermined article of taste by the user based on the output of the smell sensor.

A second aspect of the present disclosure relates to an information processing method including acquiring and estimating. The acquiring acquires the output of a smell sensor that senses a user. The estimating estimates the amount of intake of a predetermined article of taste by the user based on the output of the smell sensor.

The other aspects of the present disclosure include a program that performs the above-described information processing method and a computer readable storage medium that stores the above-described program non-transitorily.

According to the present disclosure, the amount of intake of an article of taste by the user can be estimated.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram showing an outline of an information processing system;

FIG. 2 is a diagram showing, in detail, the components of the information processing system according to a first embodiment;

FIG. 3 is a diagram showing concentration data stored in a storage unit;

FIG. 4A is a diagram showing a change in the concentration of alcohol in the air;

FIG. 4B is a diagram showing a change in the concentration of alcohol in the air;

FIG. 5 is a diagram showing an evaluation model in the first embodiment;

FIG. 6A is a diagram showing stock data stored in the storage unit;

FIG. 6B is a diagram showing stock data stored in the storage unit;

FIG. 6C is a diagram showing stock data stored in the storage unit;

FIG. 7 is a diagram showing a data flow between the modules of a control unit;

FIG. 8 is a diagram showing a positional relationship between a user and a sensor;

FIG. 9 is a flowchart showing the processing performed by the control unit in the first embodiment;

FIG. 10 is a diagram showing an evaluation model in a second embodiment;

FIG. 11 is a flowchart showing the processing performed by a control unit in the second embodiment;

FIG. 12 is a diagram showing a change in the concentration of alcohol when a room is ventilated; and

FIG. 13 is a flowchart showing the processing performed by a control unit in a third embodiment.

### DETAILED DESCRIPTION OF EMBODIMENTS

There is a system that allows a user to perform a predetermined operation to apply for the purchase of predetermined goods such as daily goods and alcoholic beverages. However, in such a system, the user has to manage the stock of, and place an order for, goods by himself or herself.

An information processing device according to this embodiment is a device that estimates the amount of intake (that is, amount of consumption) of a predetermined article of taste in order to determine when to place an order for the article of taste. More specifically, the information processing device includes a control unit configured to acquire the output of a smell sensor that senses a user and to estimate the amount of intake of a predetermined article of taste by the user based on the output of the smell sensor.

The predetermined article of taste mentioned above is an article, typically an alcoholic beverage, a cigarette, etc., that emits a peculiar smell when consumed. By acquiring the sensing result of the smell sensor that has sensed the user, the control unit can estimate, for example, that the user is drinking or smoking.

The smell sensor may be a sensor that detects any type of smell as long as the sensor can identify the strength of smell. For example, the sensor may be a sensor that determines the level of smell based on the detection result of predetermined smell molecules or may be a sensor that determines the level of alcohol smell based on the detection result of alcohol gases.

In addition, the control unit estimates the amount of intake of an article of taste by the user based on the output of the smell sensor. For example, when the user starts drinking an alcoholic beverage, the concentration of alcohol in the exhaled breath gradually rises and remains high for some time. When the user starts smoking a cigarette, the smell level in the surrounding area sharply rises and, when the user ends smoking, the smell level starts falling gradually. Therefore, by monitoring the output of the smell sensor, the control unit can estimate the amount of intake of these articles of taste by the user.

The control unit may be configured to estimate the amount of intake based on a temporal change in the concentration of smell detected by the smell sensor. For example, when the user is smoking a cigarette and the concentration of smell rises periodically, the control unit can estimate that the user is continuously smoking.

The information processing device may further include a storage unit configured to store a model for determining the amount of intake based on the temporal change in the concentration of smell. In this storage unit, a model that defines the temporal change in the concentration of smell is stored for comparison with the sensor-acquired data to make it possible to determine that the predetermined article of taste has been consumed. The model may be a machine learning model. When the temporal change in the concentration of smell is different depending upon the type of the article of taste, the information processing device may store a plurality of models in the storage unit.

The smell sensor may be a sensor that can detect the concentration of alcohol in the air, and the control unit may be configured to estimate the amount of intake of alcoholic liquor by the user based on a temporal change in the detected concentration of alcohol. The temporal change in the concentration of alcohol in the air may be used to estimate the amount of alcohol the user has drunk. In this case, the smell sensor is preferably installed in a place where the user drinks on a daily basis (for example, in the dining room).

The control unit may be configured to further acquire the information on the percentage of an alcoholic beverage the user has drunk and to estimate the amount of intake based further on the percentage of the alcoholic beverage. The higher the percentage of the alcoholic beverage the user has drunk, the faster the concentration of alcohol around the user will rise. Therefore, by further using the percentage of the alcoholic beverage the user is drinking, the control unit can estimate the amount of intake more accurately.

The smell sensor may be a sensor installed in the home of the user. Such a configuration makes it possible to estimate the amount of intake (amount of consumption) of an alcoholic beverage in the home of the user.

The control unit may be configured to further acquire the information on the state of ventilation of the home and to estimate the amount of intake based further on the information. For example, when the living room is ventilated, it is expected that the concentration of alcohol in the air will fall faster than usual. Therefore, estimating the amount of intake by further considering the state of ventilation can increase the accuracy of the estimation.

The information processing device may further store the amount of stock of an alcoholic beverage in the home of the user. In addition, the control unit may be configured to subtract the amount of intake from the stored amount of stock based on the estimated amount of intake. Such a configuration makes it possible to estimate the amount of stock of an alcoholic beverage in the home of the user.

The control unit may be configured to generate a trigger for placing an order for the alcoholic beverage when the amount of stock falls below a predetermined value. Such a configuration makes it possible to automatically place an order for the alcoholic beverage to prevent the stock of the alcoholic beverage from running out.

Embodiments of the present disclosure will be described below with reference to the drawings. The configurations of the embodiments below are exemplary and the present disclosure is not limited to the configurations of the embodiments.

### First Embodiment

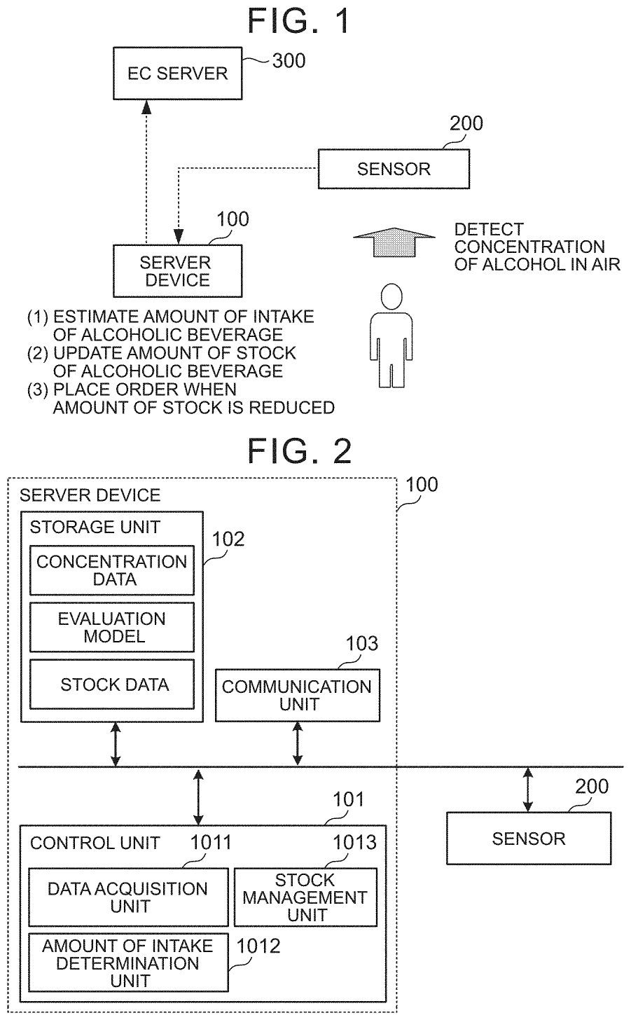

The outline of an information processing system according to a first embodiment will be described below with reference to FIG. 1. The information processing system according to this embodiment includes a server device 100 that manages the amount of stock of alcoholic beverages in the home of the user, a sensor 200 that senses the user, and an EC server 300 that accepts an order for an alcoholic beverage.

The sensor 200 is a sensor that detects the concentration of alcohol in the air. When the user drinks an alcoholic beverage, the alcohol in the exhaled breath is detected by the sensor 200. The server device 100 performs the following processing: (1) the processing for estimating the amount of intake (amount of consumption) of an alcoholic beverage by the user based on the output of the sensor 200, (2) the processing for updating the amount of stock of the alcoholic beverage in the home based on the estimation result, and (3) the processing for placing an order for the alcoholic beverage as needed. The EC server 300 is a device that accepts an order for goods (alcoholic beverages) via the network. The EC server 300 is managed by the operator of a mail-order company.

The server device 100 may be installed in the home of the user or may be installed in a remote location. One server device 100 may control a plurality of users.

FIG. 2 is a diagram showing, in more detail, the components of the information processing system according to this embodiment. First, the sensor 200 will be described. The sensor 200 includes one or more sensors that detect the concentration of alcohol in the air. The sensor 200 may be any type of sensor that can detect the exhaled breath of the user who has drunk an alcoholic beverage. Note that, when the user drinks an alcoholic beverage in a fixed place, it is preferable to install the sensor 200 near that place. For example, when the user drinks an alcoholic beverage at the dining table in the home, it is preferable to install the sensor 200 near the dining table. The sensor 200 may be composed of a plurality of sensors. For example, when the user drinks an alcoholic beverage at a plurality of places in the home, the sensor 200 may be installed at each of the plurality of places.

The server device 100 estimates the amount of intake (amount of consumption) of an alcoholic beverage by the user based on the data (hereinafter referred to as sensor data) acquired from the sensor 200. In addition, the server device 100 manages the amount of stock of alcoholic beverages in the home of the user and places an order for alcoholic beverages as needed.

The server device 100 can be configured by a general-purpose computer. That is, the server device 100 can be configured as a computer that includes a processor such as a CPU or a GPU, a main storage device such as a RAM or a ROM, and an auxiliary storage device such as an EPROM, a hard disk drive, or a removable medium. The removable medium may be, for example, a USB memory or a disc recording medium such as a CD or a DVD. The auxiliary storage device stores therein the operating system (OS), various programs, various tables, etc. Programs stored in the auxiliary storage device are loaded into the work area of the main storage device for execution. Through the execution of programs, each component is controlled to implement each function that meets the predetermined purpose that will be described later. Note that some or all of the functions may be implemented by a hardware circuit such as an ASIC or an FPGA.

A control unit 101 is an arithmetic unit that controls the operation performed by the server device 100. The control unit 101 can be implemented by an arithmetic processing unit such as a CPU. The control unit 101 includes three functional modules: a data acquisition unit 1011, an amount of intake determination unit 1012, and a stock management unit 1013. Each functional module may be implemented by causing the CPU to execute the program stored in the auxiliary storage device.

The data acquisition unit 1011 acquires sensor data from the sensor 200. The acquired sensor data indicates the concentration of alcohol in the air around the sensor 200 (i.e., around the user). When there is a plurality of sensors 200, the data acquisition unit 1011 may acquire the identifier of the sensor that has sent the data. The sensor data acquired by the data acquisition unit 1011 is sequentially accumulated in a storage unit 102, which will be described later, as concentration data. The concentration data is data indicating a temporal change in the concentration of alcohol in the air.

The amount of intake determination unit 1012 determines the amount of intake of an alcoholic beverage by the user based on the stored concentration data. More specifically, the amount of intake determination unit 1012 uses an evaluation model, which will be described later, to identify a pattern that matches the temporal change in the concentration of alcohol that has been stored and, then, identifies the amount of intake of the alcoholic beverage associated with the identified pattern. For example, the amount of intake determination unit 1012 determines that the temporal change in the concentration of alcohol, indicated by the accumulated concentration data, matches the pattern indicating that “two 350 ml cans of beer consumed”. The result of the determination is sent to the stock management unit 1013.

The stock management unit 1013 manages the data (stock data) indicating the amount of stock of an alcohol beverage in the home of the user and, based on the amount of consumption of the alcoholic beverage determined by the amount of intake determination unit 1012, updates the stock data. In addition, when the amount of stock of the alcoholic beverage falls below the predetermined value, the stock management unit 1013 places an order with the EC server 300 for the alcoholic beverage.

The storage unit 102 is configured by a main storage device and an auxiliary storage device. The main storage device is a memory where programs executed by the control unit 101 and data used by the control programs are loaded. The auxiliary storage device is a device that stores programs executed by the control unit 101 and data used by the control programs.

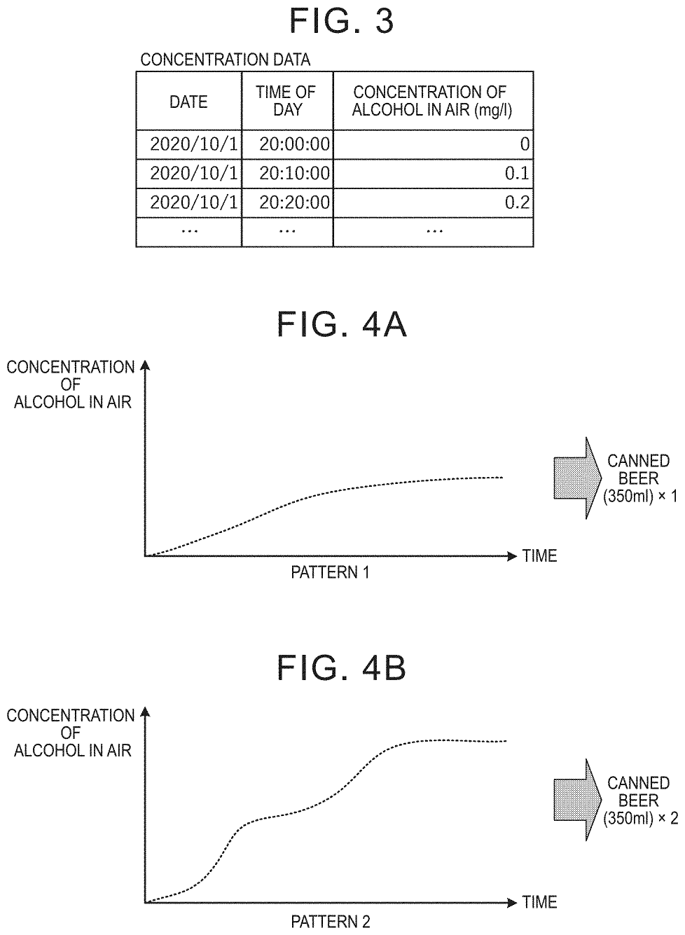

The storage unit 102 stores the concentration data described above. FIG. 3 is a diagram showing an example of concentration data. The concentration data includes the date and the time-of-day at which the sensor 200 acquired the sensor data and the concentration of alcohol in the air obtained through sensing.

In addition, the storage unit 102 stores an evaluation model for each user. The evaluation model is a model that associates a temporal change in the concentration of alcohol in the air with the amount of an alcoholic beverage consumed by the user.

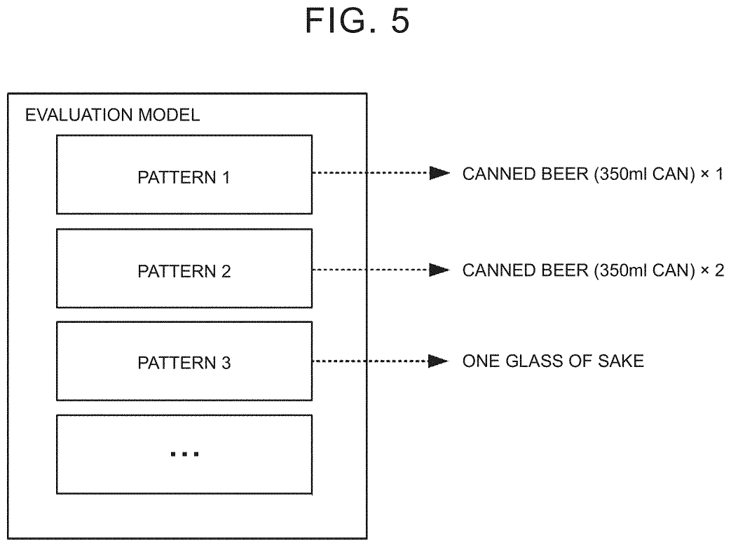

Now, the evaluation model will be described below with reference to FIG. 4A and FIG. 4B. FIG. 4A and FIG. 4B are diagrams each showing a temporal change in the concentration of alcohol in the air for each pattern of drinking by the user. For example, when the user consumes one can of beer, the concentration of alcohol in the air is assumed to change as shown in FIG. 4A. Similarly, when the user consumes two cans of beer, the concentration of alcohol in the air is assumed to change as shown in FIG. 4B. These patterns are modeled and stored for comparison with a temporal change in the concentration of alcohol in the air. This comparison makes it possible to identify which pattern the user's drinking matches.

FIG. 5 is a diagram showing the structure of an evaluation model. For example, when the temporal change in the concentration of alcohol in the air matches pattern 1, it is estimated that the user has consumed one 350 ml can of beer. In this manner, the evaluation model includes data indicating temporal changes in the concentration of alcohol in the air using a plurality of patterns. An evaluation model corresponding to the user is stored in the storage unit 102 in advance. An evaluation model may be generated, for example, by machine learning.

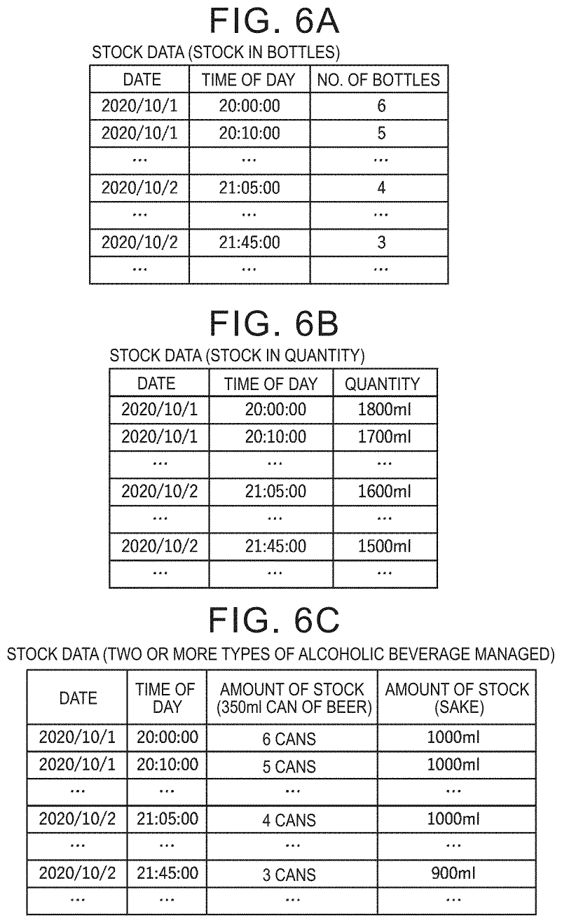

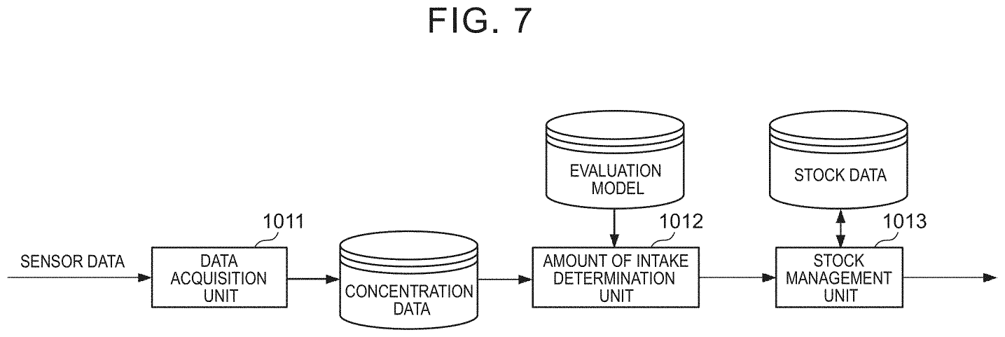

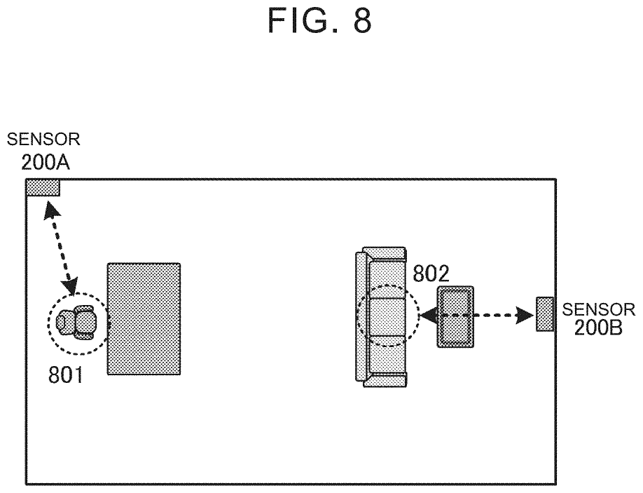

In addition, the storage unit 102 stores data (stock data) for managing the stock of alcoholic beverages in the home of the user. FIG. 6A, FIG. 6B, and FIG. 6C are diagrams each showing an example of stock data. The stock data is data that indicates, for each of the managed alcoholic beverages, the association among the amount of stock, the date, and the time of day.

The amount of stock of alcoholic beverages may be represented by type. For example, when there is a plurality of types of alcoholic beverages that the user consumes, the stock data may include the amount of stock for each type. FIG. 6A and FIG. 6B each show an example of stock data when the user consumes one type of alcoholic beverage. FIG. 6C shows an example of stock data when the user consumes a plurality of types of alcoholic beverage. The stock data is updated when the alcoholic beverage is consumed and purchased.

A communication unit 103 is a communication interface for connecting the server device 100 to the network. The communication unit 103 includes, for example, a network interface board and a wireless communication circuit for wireless communication.

The configuration shown in FIG. 2 is an example, and all or a part of the functions shown in the figure may be performed by a specifically designed circuit. Programs may be stored or executed by a combination of the main storage device and the auxiliary storage device other than those shown in the figure.

Next, the processing performed by the control unit 101 will be described with reference to FIG. 7 that is a diagram showing data sent and received between the modules.

The data acquisition unit 1011 receives sensor data from the sensor 200 installed in the home and accumulates the received sensor data in the storage unit 102 as the concentration data. As mentioned above, the concentration data is data indicating a temporal change in the concentration of alcohol in the air.

The concentration of alcohol in the air detected by the sensor 200 may be directly recorded in the concentration data. However, when the positional relationship between the user and the sensor is not fixed, it is preferable that the acquired value be corrected. FIG. 8 is a plan view showing an example of places where the user drinks alcohol in the living room. For example, assume that the user drinks alcohol in two or more places as indicated by the reference numerals 801, 802 and that the different sensors, 200A and 200B, sense that the user drinks alcohol. In this case, even when the consumed amount of alcohol is the same between these two places, the concentrations of alcohol in the air detected by the two sensors may be different. This is because the positional relationship (distance) between the sensor and the user is different between the two places. Therefore, when two or more sensors 200 are used in the home, it is preferable for each sensor to use its own standard to convert the concentration of alcohol in the air into a value that can be used for comparison. For example, the concentration of alcohol in the exhaled breath of the user may be estimated based on the concentration of alcohol in the air and the estimated value may be stored as the concentration data.

The amount of intake determination unit 1012 determines the user's consumption of the alcoholic beverage in the most recent predetermined period based on the acquired concentration data. More specifically, the amount of amount of intake determination unit 1012 determines which of the multiple patterns, defined in the evaluation model, matches the temporal change in the concentration of alcohol in the air indicated by the concentration data and, based on the matched pattern, acquires the amount of intake of the alcoholic beverage associated with that pattern. For example, when the temporal change in the concentration of alcohol in the air matches the pattern shown in FIG. 4A, the amount of intake determination unit 1012 determines that the user has drunk one 350 ml can of beer.

A machine learning model may be used as an evaluation model. For example, by executing machine learning with a temporal change in the concentration of alcohol as the input data and with the amount of consumption of an alcoholic beverage as the training data, a machine learning model can be obtained that estimates the amount of consumption of the alcoholic beverage based on the concentration data.

The pattern matching/non-matching method is not limited to the method described above. For example, whether the temporal change in the concentration of alcohol matches a predetermined pattern may be determined based on the duration of drinking, based on when and how often the numerical concentration value increases, or based on the decrease in the numerical concentration value.

The determination result is sent to the stock management unit 1013. When one type of alcoholic beverage is managed as the stock, the amount of intake of the alcoholic beverage (for example, “350 ml can×1”) is sent as the determination result. When a plurality of types of alcoholic beverages is managed as the stock, the type and the amount of intake (for example, “canned beer/350 ml can×1”) are sent.

The stock management unit 1013 updates the stock data based on the information sent from the amount of intake determination unit 1012. More specifically, the stock management unit 1013 subtracts the amount of intake from the current amount of stock and adds a new record to the stock data in the storage unit 102. When a plurality of types of alcoholic beverage is managed as the stock, the stock management unit 1013 subtracts the amount of intake from the amount of stock of the corresponding type. In addition, when the amount of stock of an alcoholic beverage falls below the predetermined level, the stock management unit 1013 performs processing for placing an order for the alcohol beverage. For example, the stock management unit 1013 generates data for placing an order for the predetermined quantity of the alcoholic beverage of the predetermined type and sends the generated data to predetermined EC server 300.

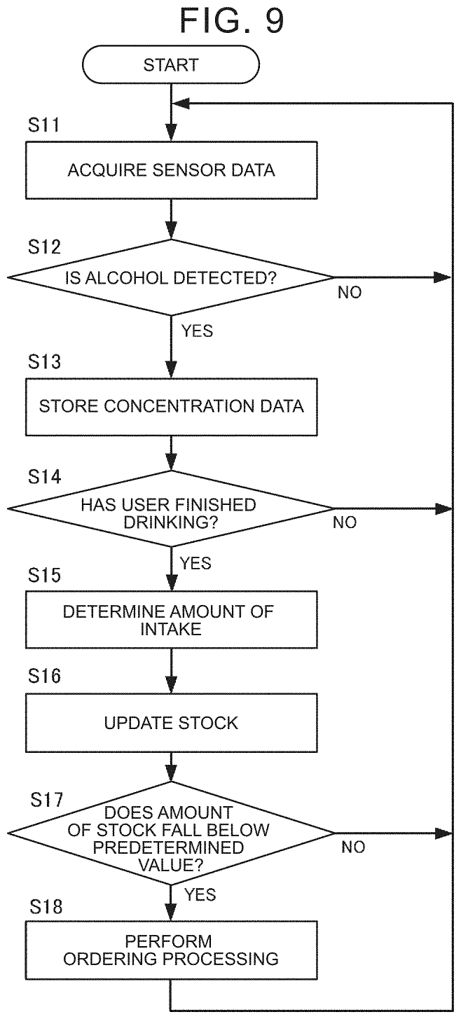

FIG. 9 is a flowchart showing the processing performed by the server device 100. The processing shown in the figure is periodically performed while the server device 100 is in operation

First, in step S11, the data acquisition unit 1011 acquires sensor data from the sensor 200. Next, in step S12, the data acquisition unit 1011 determines whether alcohol is detected based on the acquired sensor data. When alcohol is not detected, i.e., when the concentration of alcohol in the air is zero (or substantially zero), the processing returns to step S11. When alcohol is detected in the air, the processing proceeds to step S13.

In step S13, the data acquisition unit 1011 generates a new record of concentration data based on the acquired sensor data and adds the generated record to the storage unit 102 as the concentration data.

Next, in step S14, the data acquisition unit 1011 determines whether the user has finished drinking. Whether the user has finished drinking may be determined based on the sensor data. For example, when the detected concentration of alcohol falls below the predetermined value, the data acquisition unit 1011 may determine that the user has finished drinking. In addition, whether the user has finished drinking may be determined using a sensor other than that used in the example. For example, when it is determined, based on the output of the motion sensor or the image sensor, that the user has left the dining table, the data acquisition unit 1011 may determine that the user has finished drinking. Furthermore, in a situation where the opening/closing of the refrigerator door can be sensed, the data acquisition unit 1011 may determine that the user has finished drinking when the refrigerator door is not opened or closed for a predetermined time or more. When it is determined that the user has finished drinking, the processing proceeds to step S15. Otherwise, the processing proceeds to step S11.

In step S15, the amount of intake determination unit 1012 determines the amount of intake of the alcoholic beverage by the user. In this step, the amount of intake determination unit 1012 uses the evaluation model stored in the storage unit 102 to determine the type and the amount of intake of the alcoholic beverage. The determination result is sent to the stock management unit 1013.

In step S16, the stock management unit 1013 updates the stock data based on the result of the determination made by the intake determination unit 1012.

In step S17, the stock management unit 1013 checks the managed alcoholic beverages and determines whether the amount of stock of any of the alcoholic beverages falls below the predetermined value. When the amount of stock of any of the alcoholic beverages falls below the predetermined value, the stock management unit 1013 generates data (ordering data) for placing an order for that beverage and sends the generated ordering data to the EC server 300 (step S18). When the amount of stock is not smaller than the predetermined value, the processing returns to step S11.

As described above, the system in the first embodiment estimates the amount of consumption of an alcoholic beverage by the user based on the concentration of alcohol in the air detected by the sensor. In addition, the system estimates the stock based on the estimated amount of consumption and autonomously places an additional order. This configuration eliminates the need for the user to manage the stock of an alcoholic beverage by himself or herself and to determine when to place an order for the alcoholic beverage, thus improving usability.

### Second Embodiment

In the first embodiment, the amount of consumption of an alcoholic beverage is estimated based on the temporal change in the concentration of alcohol in the air. Meanwhile, the temporal change in the concentration of alcohol in the air depends on the alcohol percentage of the alcoholic beverage that has been consumed. Therefore, when there is a plurality of alcoholic beverages each with a different percentage, it is sometimes impossible to accurately determine what has been consumed. For example, when the temporal change in the concentration of alcohol in the air is the same, it may not be possible to determine whether the user has drunk a large amount of low percentage alcohol or the user has drunk a small amount of high percentage alcohol.

To address this problem, the information about the type or percentage of the alcoholic beverage the user has drunk is used in a second embodiment as the additional information.

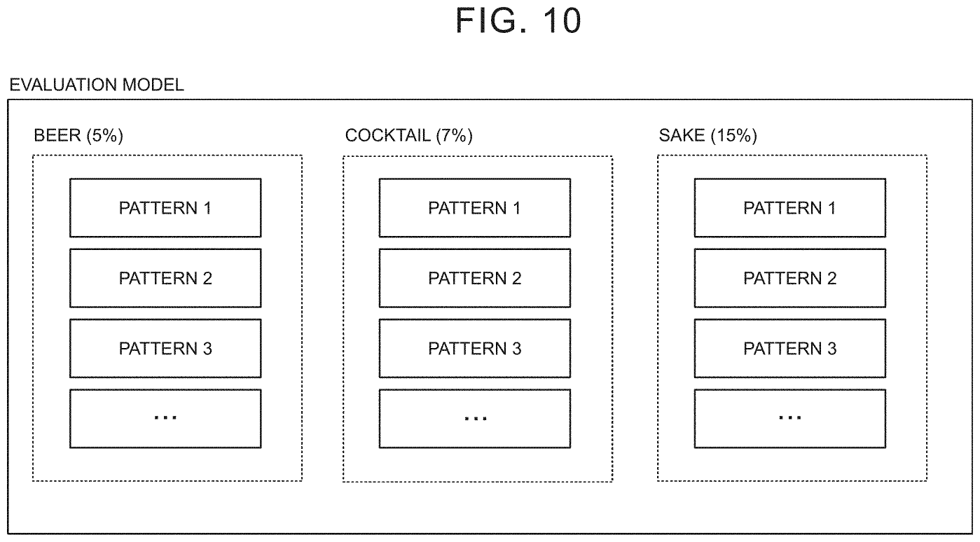

In the second embodiment, the evaluation model stores a plurality of patterns for each of the types or percentages of alcoholic beverages the user has drunk. FIG. 10 is a diagram showing an example of an evaluation model in the second embodiment. In this example, a plurality of patterns is defined for each of a plurality types of beverages such as beer with an alcohol percentage of 5, cocktail with an alcohol percentage of 7, and sake with an alcohol percentage of 15.

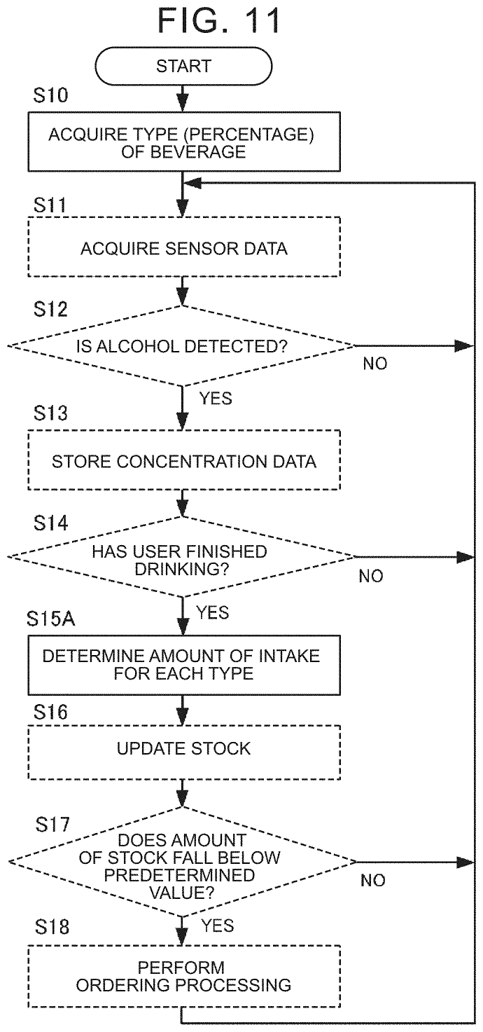

FIG. 11 is a flowchart showing the processing performed by the control unit 101 in the second embodiment. The steps indicated by the dotted lines are the same as those in the first embodiment and, therefore, the detailed description thereof will be omitted. In step S10, the amount of intake determination unit 1012 acquires the type and/or percentage of the alcoholic beverage the user is drinking. The type and/or percentage of the alcoholic beverage the user is drinking may be determined, for example, based on the result obtained by sensing the user. The processing in steps S11 to S14 is the same as that in the first embodiment.

In step S15A, the amount of intake determination unit 1012 extracts data, corresponding to the type and/or percentage that have been determined, from the stored evaluation model and, using the same method used in the first embodiment, estimates the amount of intake of the alcoholic beverage. The processing in steps S16 to S18 is the same as that in the first embodiment.

According to the second embodiment, even when there is a plurality of types of alcoholic beverage each having a different alcohol percentage, the amount of consumption can be estimated accurately.

### Third Embodiment

In the first and second embodiments, the concentration of alcohol in the air in a room is detected. Meanwhile, when an air conditioner or a ventilator is operating in the room, the strength of smell is reduced faster than usual and, therefore, the concentration of alcohol cannot be determined accurately in some cases. To address this problem, the concentration of alcohol is corrected in a third embodiment based on the operating condition of the air conditioner (ventilator) in the room.

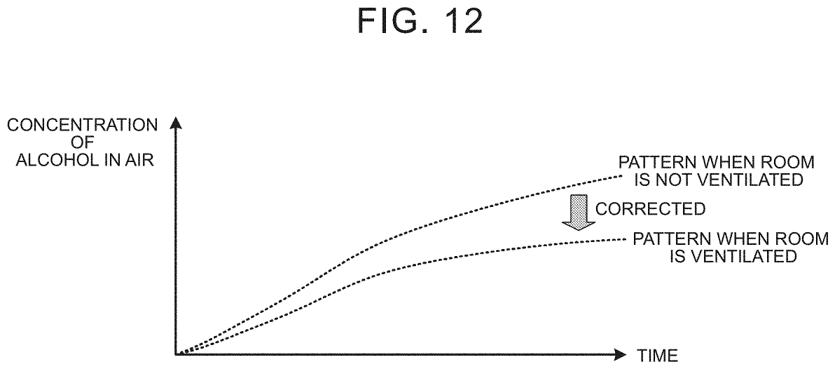

FIG. 12 is a graph showing a change in the concentration of alcohol in the air. As shown in the figure, when the air conditioner is operating for ventilation, the alcohol component in the air is discharged to the outside and, therefore, the concentration of alcohol is quickly reduced. To address this problem, when the air conditioner is operating for ventilation, it is preferable to correct the patterns used for comparison.

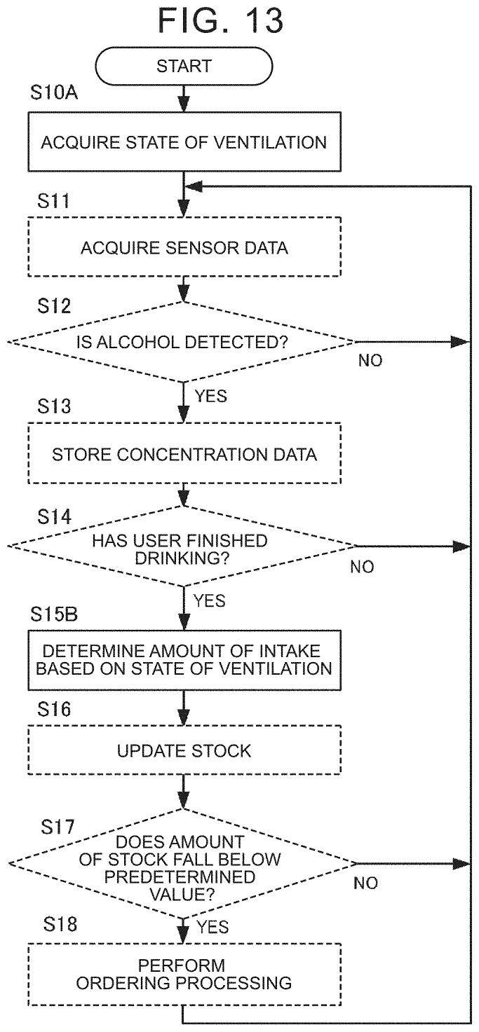

FIG. 13 is a flowchart showing the processing performed by the control unit 101 in the third embodiment. The steps indicated by the dotted lines are the same as those in the first embodiment and, therefore, the detailed description thereof will be omitted. In step S10A, the amount of intake determination unit 1012 acquires, from a predetermined device, the state of ventilation (or state of air conditioning with ventilation) in the room where the sensor 200 is installed. The predetermined device may be, for example, a home server that controls air conditioning or ventilation in the home, or may be the controller of the air conditioner or the ventilation fan. The processing in steps S11 to S14 is the same as that in the first embodiment.

In step S15B, the amount of intake determination unit 1012 estimates the amount of intake of the alcoholic beverage in the same manner as in step S15 but in a manner different from that in the first embodiment or the second embodiment in that the pattern included in the evaluation model is corrected based on the state of ventilation determined in step S10A. The correction may be performed, for example, by multiplying the concentration of alcohol by a predetermined coefficient that is determined depending upon the strength of ventilation. Furthermore, when the ventilation volume can be obtained, the reduction in the concentration of alcohol may be simulated according to the ventilation volume and, based on the simulation result, the pattern may be overwritten. The processing in steps S16 to S18 is the same as that in the first embodiment.

According to the third embodiment, the amount of consumption of an alcoholic beverage can be estimated even when a ventilator is operating in the room.

### Modifications

The above embodiments are merely exemplary and the present disclosure may be modified as necessary for implementation without departing from the spirit thereof. For example, the processing and units described in the present disclosure can be carried out in any combination as long as there is no technical contradiction.

In the description of the embodiments, though an alcoholic beverage is assumed as the article of taste, the present disclosure may be applied also to cigarettes and the like that emit smell when consumed. In this case, too, an evaluation model, such as that described above, may be used to determine the number of smoked cigarettes.

Although a sensor that detects alcohol in the air is used as the sensor 200 in the description of the embodiments, a sensor (smell sensor) capable of detecting a plurality of types of smell-causing molecules may also be used. When using such a smell sensor, it is necessary to detect only the smell of alcohol since there are various smell-causing molecules. Therefore, in this case, it is preferable to add a unit or a logic specifically designed for extracting the level of smell corresponding to alcohol based on the output of the sensor 200.

The processing described as being performed by one device may be divided for execution by a plurality of devices. Conversely, the processing described as being performed by different devices may be performed by one device. In a computer system, the hardware configuration (server configuration) for implementing each function may be flexibly changed.

The present disclosure can also be implemented by supplying a computer program, which implements the functions described in the above embodiments, to a computer so that one or more processors of the computer can read and execute the program. Such a computer program may be provided to the computer by a non-transitory computer-readable storage medium that can be connected to the system bus of the computer or may be provided to the computer via a network. The non-transitory computer-readable storage medium includes any type of disk, such as a magnetic disk (floppy (registered trademark) disk, hard disk drive (HDD), etc.) and an optical disc (CD-ROM, DVD disc, Blu-ray disc, etc.), and any type of medium suitable for storing electronic instructions such as a read only memory (ROM), a random access memory (RAM), an EPROM, an EEPROM, a magnetic card, a flash memory, and an optical card.

What is claimed is:

1. An information processing device comprising a control unit configured to acquire an output of a smell sensor that senses a user and to estimate an amount of intake of a predetermined article of taste by the user based on the output of the smell sensor.

2. The information processing device according to claim 1, wherein the control unit is configured to estimate the amount of intake based on a temporal change in a concentration of smell detected by the smell sensor.

3. The information processing device according to claim 2, the information processing device further comprising a storage unit configured to store a model for determining the amount of intake based on the temporal change in the concentration of smell.

4. The information processing device according to claim 1, wherein
the smell sensor is a sensor configured to detect a concentration of alcohol in the air, and
the control unit is configured to estimate an amount of intake of alcoholic liquor by the user based on a temporal change in the detected concentration of alcohol.

5. The information processing device according to claim 4, wherein the control unit is configured to further acquire information on a percentage of an alcoholic beverage the user has drunk and to estimate the amount of intake based further on the percentage of the alcoholic beverage.

6. The information processing device according to claim 4, wherein the smell sensor is a sensor installed in a home of the user.

7. The information processing device according to claim 6, wherein the control unit is configured to further acquire information on a state of ventilation of the home and to estimate the amount of intake based further on the information.

8. The information processing device according to claim 6, the information processing device further comprising a storage unit configured to further store an amount of stock of an alcoholic beverage in the home of the user.

9. The information processing device according to claim 8, wherein the control unit is configured to subtract the amount of intake from the stored amount of stock based on the estimated amount of intake.

10. The information processing device according to claim 9, wherein the control unit is configured to generate a trigger for placing an order for the alcoholic beverage when the amount of stock falls below a predetermined value.

11. An information processing method comprising:
acquiring an output of a smell sensor that senses a user; and
estimating an amount of intake of a predetermined article of taste by the user based on the output of the smell sensor.

12. The information processing method according to claim 11, wherein the estimating estimates the amount of intake based on a temporal change in a concentration of smell detected by the smell sensor.

13. The information processing method according to claim 12, the information processing method further comprising acquiring a model for determining the amount of intake based on the temporal change in the concentration of smell.

14. The information processing method according to claim 11, wherein
the smell sensor is a sensor that detects a concentration of alcohol in the air, and
the estimating estimates an amount of intake of alcoholic liquor by the user based on a temporal change in the detected concentration of alcohol.

15. The information processing method according to claim 14, wherein the estimating further acquires information on a percentage of an alcoholic beverage the user has drunk and estimates the amount of intake based further on the percentage of the alcoholic beverage.

16. The information processing method according to claim 14, wherein the smell sensor is a sensor installed in a home of the user.

17. The information processing method according to claim 16, wherein the estimating further acquires information on a state of ventilation of the home and estimates the amount of intake based further on the information.

18. The information processing method according to claim 16, the information processing method further comprising acquiring an amount of stock of an alcoholic beverage in the home of the user.

19. The information processing method according to claim 18, wherein the estimating generates a trigger for placing an order for the alcoholic beverage based on the acquired amount of stock and the estimated amount of intake.

20. A program causing a computer to execute the information processing method according to claim 11.
